(19) United States
(12) Patent Application Publication
(10) Pub. No.: US 2023/0165765 A1
(43) Pub. Date: Jun. 1, 2023

(54) OPTICALLY-TRANSFORMING NAIL COATINGS, METHODS, AND SYSTEMS

(71) Applicant: L'OREAL, Paris (FR)

(72) Inventors: Charles E WARD, JR., BURLINGTON, NJ (US); I-Fan HSIEH, SCOTCH PLAINS, NJ (US); Luke Zachary SHAPIRO, MATAWAN, NJ (US)

(73) Assignee: L'OREAL, PARIS (FR)

(21) Appl. No.: 17/537,553

(22) Filed: Nov. 30, 2021

(51) Int. Cl. A61K 8/25; A61Q 3/02

(52) U.S. Cl. CPC A61K 8/25; A61Q 3/02; A61K 2800/436; A61K 2800/95

(57) ABSTRACT

Compositions, kits and methods of transforming the color appearance of a nail are provided. Methods include applying a color-transforming composition to a nail having a decorative coating with substantially uniform color appearance formed thereon. The color-transforming composition comprises solvent; a color system comprising effect colorants; a suspending system comprising hydrophilic silica; and a film-forming system, wherein the color-transforming composition is substantially free of clays.

### FIELD OF THE INVENTION

The present invention relates to nail compositions that optically transform a coated nail surface as well as to systems and methods for transforming the appearance of a coated nail surface.

### DISCUSSION OF THE BACKGROUND

Consumers enjoy the use of nail polish to create color effects on their nails for fashion, beauty and self-confidence. Nail polishes or coatings are indeed well-known for use to impart color and optical effects on the nails. This can be accomplished using various coatings that include one or more colorants. It is also known to apply certain protective or gloss-enhancing, transparent “topcoat” coatings on a nail surface having a color coat formed thereon. However, the present inventors have recognized that for reasons of convenience and fashion, there remains a need to easily and readily alter the perceived color properties of a nail that has a color coating already formed thereon. The present inventors have indeed now found that it is possible to transform the color appearance of a nail having certain coating(s) formed thereon.

### SUMMARY OF THE INVENTION

The present invention relates to a method of making up the nails or transforming the color appearance of nails. The nail has a decorative coating formed thereon having substantially uniform color appearance. The method includes applying a color-transforming composition to the nail that has the decorative coating having substantially uniform color appearance formed thereon. The color-transforming composition includes (1) solvent; (2) a color system; (3) a suspending system; and (4) a film-forming system.

The color system of the color-transforming composition includes one or more colorants, the one or more colorants comprising one or more effect colorants, wherein the one or more effect colorants are present in the color-transforming composition in a concentration by weight from about 0.1% to about 2%. The color system optionally includes one or more additional colorants. The color system has a colorant fraction of effect colorants that is at least about 50% by weight (i.e., the majority of colorants in the color-transforming composition are effect colorants). The suspending system includes one or more suspending agents, the one or more suspending agents comprising hydrophilic silica. The color-transforming composition is substantially free of clays.

According to certain embodiments, the decorative coating that has been formed on the nail is formed by applying a nail polish composition onto the nail. The nail polish composition may have from about 0.5% by weight to about 5% by weight of colorants and may have a colorant fraction of effect colorants that is less than about 10% by weight, such as less than about 1% by weight, such as less than about 0.25% by weight. According to certain embodiments the one or more effect colorants comprise or consist of pearlescent pigments.

According to certain other aspects, a nail composition that may be used to transform color appearance includes (1) solvent; (2) a color system; (3) a suspending system; and (4) a film-forming system.

The color system includes one or more pigments, the one or more colorants comprising one or more pearlescent pigments, wherein the one or more pearlescent pigments are present in the color-transforming composition in a concentration by weight from about 0.1% to about 2%. The color system has a colorant fraction of pearlescent pigments that is at least about 90% by weight. The suspending system includes one or more suspending agents, the one or more suspending agents comprising hydrophilic silica. The suspending system is at least about 80% hydrophilic silica. The nail composition is substantially free of clays. The nail composition includes from about 50% to about 85% by weight of solvents.

### DETAILED DESCRIPTION OF THE INVENTION

As used herein, the expression “at least one” means one or more and thus includes individual components as well as mixtures/combinations.

“Film former” or “film forming agent” as used herein means a polymer or resin that leaves a film on the substrate to which it is applied, for example, after a solvent accompanying the film former has evaporated, absorbed into and/or dissipated on the substrate.

“Makeup Result” as used herein, refers to compositions where color remains the same or substantially the same as at the time of application, as viewed by the naked eye, after an extended period of time. “Makeup Result” may be evaluated by evaluating color appearance may be evaluated by a test involving the application of a composition to nails and evaluating the color of the composition after a period of time. For example, the color appearance of a composition may be evaluated immediately following application to nails and these characteristics may then be re-evaluated and compared after a certain amount of time. Further, these characteristics may be evaluated with respect to other compositions, such as commercially available compositions.

“Making up” as used herein means to provide decoration (for example, color) to the nail.

“Adhesion” as used herein, refers to chemical or physical bonding between a coating and a substrate. Good adhesion between nail polish and nail surface should translate to good wear properties on consumers. Adhesion properties can be quantified by in-vitro method such as a cross-cut adhesion test. In the test, a lattice pattern is cut into the coating and penetrates through to the substrate. A pressure sensitive tape is applied to the sample and then pulled off. The adhesion property can be quantified by the area of the coating remaining after peeling. For example, if the whole film remains after peeling, it indicates excellent adhesion. If most of the film gets peeled off, it indicates poor adhesion. The cross-cut test is an industrial standard test for testing adhesion for coatings. (Reference #ISO/DIN 2409, ASTM D3359).

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Nail” as used herein includes fingernails as well as toenails.

“Transform the color appearance” refers to the ability to change the color and/or color uniformity such as that of a decorative coating (also referred to herein as “first coating”) having substantially uniform color appearance that is formed on a nail. The transformation may be from that of a substantially uniform color to one that is nonuniform and which may vary as a function of the conditions of observation, such as angle of observation.

“Volatile”, as used herein, means having a flash point of less than about 100° C.

“Non-volatile”, as used herein, means having a flash point of greater than about 100° C.

“Substantially free” as it is used herein means that while it is preferred that no amount of the specific component be present in the composition, it is possible to have very small amounts of it in the compositions of the invention provided that these amounts do not materially affect at least one, preferably most, of the advantageous properties of the conditioning compositions of the invention. In certain embodiments, substantially free means less than about 2% of the identified ingredient, such as less than about 1%, such as less than about 0.5%, such as less than about 0.1% of the ingredient. The term “anhydrous” means substantially free of water.

Other than in the operating examples, or where otherwise indicated, all numbers expressing quantities of ingredients and/or reaction conditions are to be understood as being modified in all instances by the term “about,” meaning within 10% to 15% of the indicated number. For example, a concentration by weight of about 60% means a concentration by weight that is within 15% of 60, i.e., from 51% to 69%, such as within 10% of 60, i.e., from 54% to 66%.

Numerical ranges are inclusive of endpoints and meant to include all combinations and sub-combinations. For example, from about 5%, 10% or 15% to about 20%, 50% or 60% means about 5% to about 20%, about 5% to about 50%, about 5% to about 60%, about 10% to about 20%, about 10% to about 50%, about 10% to about 60%, about 15% to about 20%, about 15% to about 50%, or about 15% to about 60%.

All concentrations in this specification are by weight unless otherwise specifically stated differently.

“Actives basis” as used herein means considering only the particular component of ingredient (e.g., in a composition) and ignoring other chemically unrelated components that may be also be present in the same raw material source of that particular component.

“Solids basis” as used herein means considering only the solids components including coatings and absorbed materials such as dyes (e.g., in a composition) and ignoring volatile solvents and water. Fractions of effect colorants, as described herein are calculated on a solids basis.

“Polymer” as used herein refers to a compound having molecules that comprise repeat units. In certain embodiments, polymer useful for embodiments described herein have a repeat unit comprising carbon and hydrogen, such as repeat units comprising carbon, hydrogen and optionally oxygen or nitrogen.

The compositions, coats and methods of the present invention can comprise, consist of, or consist essentially of the essential elements and limitations of the invention described herein, as well as any additional or optional ingredients, components, or limitations described herein or otherwise useful. For example, the compositions and coats can “consist essentially of” or “consist of” effect colorants (such as pearl pigments) as the coloring agent.

The compositions and methods of the present invention can “comprise,” “consist of” or “consist essentially of” the essential elements and limitations of the invention described herein, as well as any additional or optional ingredients, components, or limitations described herein or otherwise useful. For purposes of the compositions and methods of the present invention where the invention “consists essentially of” the identified ingredients and/or process steps, the sole “basic and novel property” of such compositions and/or methods is the ability to transform the color appearance of a decorative coating having substantially uniform color appearance that is formed on a nail. Further, given that it is contemplated that other colorants can be added to the color-transforming composition in the context of the present invention, a “material effect” on the basic and novel property of the invention can only be an adverse effect. That is, because positive effects on color-transforming properties (such as those influenced by effect colorants, such as pearlescent pigments) are within the scope of the present invention, only ingredients which have a material adverse effect on the properties of transforming color appearance provided by effect pigments would be relevant to determining whether or not compositions or methods “consist essentially of” the required elements.

Decorative Coating Having Substantially Uniform Color Appearance

According to aspects of the present invention, a nail has a decorative coating (also known as “nail polish” or nail enamel”) formed thereon. The decorative coating has a substantially uniform color appearance. By “substantially uniform color appearance,” it is meant that while the coating on the nail may have some non-uniformities in gloss or thickness, from a distance of two feet, the nail surface will generally have (such as across distances of at least a few millimeters, preferably across substantially the entire nail)) an appearance of uniform, non-transparent color. The uniform color may be any of various colors commonly used for nail coatings (e.g., a shade of red, pink, brown, blue, gray, black, white, among others).

According to certain embodiments, the decorative coating comprises one or more colorants substantially uniformly distributed throughout decorative coating. The one or more colorants present in the decorative coating are selected from various ones described in this specification. The one or more colorants present in the decorative coating include a fraction of conventional colorants and optionally a fraction of effect colorants (each of these terms are described in this specification, below). According to certain embodiments, the fraction of effect colorants as compared to a total concentration of colorants in the decorative coating (i.e., the “colorant fraction of effect colorants” in the decorative coating) is less than about 10%, such as less than about 5%, such as less than about 1%, such as less than about 0.5%, such as less than about 0.25%, such as 0%. For clarity, the colorant fraction of effect colorants is calculated by dividing the total concentration by weight of effect colorants by the total concentration of all colorants in the coating. The concentrations used to determine colorant fractions of effect colorants are all calculated on a solids basis.

The decorative coating formed on the nail may be formed for example by applying a conventional nail polish composition (“color coat”) having colorants (e.g., the colorant fractions of effect colorants) as described in the paragraph above—either directly to the nail or for example over a transparent basecoat. In certain embodiments, the nail polish composition will include from about 0.5% or 1% to about 3% or 5% by weight of colorants on a solids basis; and may include about 50% to about 85% solvents, with the balance being other solids such as film formers and the like. A conventional transparent topcoat may be optionally formed over the color coat. The various coatings are generally allowed to dry (by solvent evaporation) or cure (for example, by radiation such as ultraviolet radiation) before applying additional coatings.

Color-Transforming Composition

According to certain embodiments of the invention, a color-transforming composition is applied to the nail having the decorative coating having substantially uniform color appearance formed thereon. The color-transforming composition includes (1) solvent; (2) a color system; (3) a suspending system; and (4) a film-forming system.

Solvent

The color-transforming composition includes one or more solvents. According to preferred embodiments, compositions of the invention comprise at least one solvent. Suitable examples of solvents, include, but are not limited to, organic solvents such as ketones which are liquid at room temperature such as, for example, methyl ethyl ketone, methyl isobutyl ketone, diisobutyl ketone, isophorone, and acetone; alcohols which are liquid at room temperature, such as ethanol, isopropanol, n-propanol, n-butanol, diacetone alcohol, and 2-butoxyethanol; glycols which are liquid at room temperature, such as propylene glycol, pentylene glycol, and glycerol; propylene glycol ethers which are liquid at room temperature, such as propylene glycol monomethyl ether, propylene glycol monomethyl ether acetate, and dipropylene glycol mono-n-butyl ether; short-chain esters (having from 3 to 8 carbon atoms in total) such as ethyl acetate, methyl acetate, propyl acetate, n-butyl acetate, and isopentyl acetate; ethers which are liquid at room temperature; alkanes which are liquid at room temperature such as decane, heptane, dodecane, and isododecane; aromatic cyclic compounds which are liquid at room temperature. According to certain embodiments, the solvent is selected from ethyl acetate, propyl acetate, butyl acetate, isopropanol and ethanol, and combinations thereof. The solvent preferably comprises from about 10% to about 95% by weight, preferably from about 30% to about 90% by weight, and preferably from about 50% to about 85%, by weight, relative to the total weight of the composition.

Color System

The color-transforming includes a color system consisting of one or more colorants. Suitable colorants (coloring agents) include any colorant typically found in nail compositions. Suitable colorants include conventional colorants and effect colorants.

In accordance with preferred embodiments of nail compositions providing color to a nail, the colorants are preferably present in a total amount sufficient to provide color to the nail, preferably in an amount of from about 0.1% to about 4% by weight, preferably from about 0.25% to about 3% by weight, and preferably from about 0.5 to about 2% by weight, of the total weight of the composition, including all ranges and subranges therebetween.

Suitable conventional colorants include but are not limited to, lipophilic dyes, conventional pigments, and their mixtures.

Suitable examples of lipophilic dyes are, for example, Sudan red, DC Red 17, DC Green 6, β-carotene, soybean oil, Sudan brown, DC Yellow 11, DC Violet 2, DC Orange 5 and quinoline yellow.

Suitable conventional pigments can be white or colored, inorganic and/or organic and coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Mention may also be made, among organic pigments, of carbon black, pigments of D & C type and lakes based on cochineal carmine or on barium, strontium, calcium or aluminum, such as D&C Red No. 10, 11, 12, and 13, D&C Red No. 7, D&C Red No. 5 and 6, and D&D Red No. 34, as well as lakes such as D&C Yellow Lake No. 5 and D&C Red Lake No. 2. The conventional pigments may further include rosin surface treatments (i.e., they are rosinated colorants).

In accordance with preferred embodiments, the conventional colorants are optionally present in the color-transforming composition in an amount from about 0% to about 2% by weight, preferably from about 0% to about 1% by weight, preferably from about 0% to about 0.5%, such as from about 0% to about 0.2%, of the total weight of the composition, including all ranges and subranges therebetween.

Effect Colorant

“Effect colorant” is understood to mean pigments which create in a general way a colored appearance (characterized by a certain shade, a certain vividness and a certain brightness) which is nonuniform and which changes as a function of the conditions of observation (light, temperature, angles of observation, and the like). They thereby contrast with conventional colorants, which provide a conventional uniform opaque, semitransparent or transparent shade.

Several types of effect colorants exist: those having a low refractive index, such as fluorescent or photochromic pigments, and those including a higher refractive index, such as pearlescent agents (also referred to herein as “pearlescent pigments” or “pearl pigments”), interference pigments or glitters.

According to certain embodiments, the effect colorant is chosen from a pearlescent pigment. Pearlescent pigments typically include a substrate material that may be in the form of a plate-shape having a face dimension from about 4 to 1000 microns, such as about 1 to 150 microns, and a thickness from about 0.2 microns to about 1 micron. The substrate may comprise or consist of a synthetic mica such as a fluorophlogopite mineral, natural mica, silica, a borosilicate or titanium dioxide. One or more coatings, such as those having a thickness from about 40 nm to about 165 nm are formed on the substrate. The coatings, at least some of which are chemically different (and, for example, higher refractive index) than the substrate, may include metal oxides such as iron oxide, tin oxide, or titanium dioxide; silica, and combinations thereof. The coatings may additionally include one or more dyes or pigments such as a ferrocyanide or carbon black.

Mention may be made, as examples of pearlescent pigments, such as mica coated with titanium or with bismuth oxychloride, colored pearlescent pigments, such as mica covered with titanium and with iron oxides, mica covered with iron oxide, mica covered with titanium and in particular with ferric blue or with chromium oxide, mica covered with titanium and with an organic pigment as defined above, and also pearlescent pigments based on bismuth oxychloride. Mention may be made, as pearlescent pigments, of the pearlescent agents Cellini sold by BASF (mica-TiCh-lake), Prestige sold by Eckart (mica-TiCk), Prestige Bronze sold by Eckart (mica-FeiCh) or Colorona sold by Merck (mica-TiCk-FeiCh).

Mention may also be made of gold-colored pearlescent agents sold in particular by BASF under the names Brilliant Gold 212G (Timica), Gold 222C (Cloisonne), Sparkle Gold (Timica), Gold 4504 (Chromalite) and Monarch Gold 233X (Cloisonne); bronze pearlescent agents sold in particular by Merck under the names Bronze Fine (17384) (Colorona) and Bronze (17353) (Colorona) and by BASF under the name Super Bronze (Cloisonne); orange pearlescent agents sold in particular by BASF under the names Orange 363C (Cloisonne) and Orange MCR 101 (Cosmica) and by Merck under the names Passion Orange (Colorona) and Matte Orange (17449) (Microna); brown-colored pearlescent agents sold in particular by BASF under the names Nu-Antique Copper 340XB (Cloisonne) and Brown CL4509 (Chromalite); pearlescent agents with a copper glint sold in particular by BASF under the name Copper 340A (Timica); pearlescent agents with a red glint sold in particular by Merck under the name Sienna Fine (17386) (Colorona); pearlescent agents with a yellow glint sold in particular by BASF under the name Yellow (4502) (Chromalite); red-colored pearlescent agents with a gold glint sold in particular by BASF under the name Sunstone GO 12 (Gemtone); pink pearlescent agents sold in particular by BASF under the name Tan Opale G005 (Gemtone); black pearlescent agents with a gold glint sold in particular by BASF under the name Nu-Antique Bronze 240 AB (Timica), blue pearlescent agents sold in particular by Merck under the name Matte Blue (17433) (Microna), white pearlescent agents with a silvery glint sold in particular by Merck under the name Xirona Silver, and golden green pinkish orangey pearlescent agents sold in particular by Merck under the name Indian Summer (Xirona), and mixtures thereof.

Mention may also be made, still as examples of pearlescent agents, of particles comprising a borosilicate substrate coated with titanium oxide. Particles having a glass substrate coated with titanium oxide are sold in particular under the name Metashine MC1080RY by Toyal.

Additionally, mention may also be made, as examples of other effect pigments, of polyethylene terephthalate glitter, in particular that sold by Meadowbrook Inventions under the name Silver IP 0.004×0.004 (silver glitter). It is also possible to envisage multilayer pigments based on synthetic substrates, such as alumina, silica, calcium sodium borosilicate or calcium aluminum borosilicate and aluminum.

The effect pigments can also be chosen from reflective particles, that is to say in particular particles having a size, a structure, in particular a thickness of the layer or layers of which it is composed and their physical and chemical nature, and the surface condition which allow them to reflect incident light. This reflection may, if appropriate, have an intensity sufficient to create, at the surface of the composition or of the mixture, when the latter is applied to the support to be made up, highlight points visible to the naked eye, that is to say more luminous points which contrast with their environment by appearing to sparkle.

The reflective particles can be selected so as not to detrimentally affect, to a significant extent, the coloring effect generated by the coloring agents which are combined with them and more particularly so as to optimize this effect in terms of color rendition. They can more particularly have a yellow, pink, red, bronze, orangey, brown, gold and/or coppery color or glint.

These particles can exhibit varied forms and can in particular be in the platelet or globular form, especially the spherical form.

The reflective particles, whatever their form, may or may not exhibit a multilayer structure and, in the case of a multilayer structure, may exhibit, for example, at least one layer of uniform thickness, in particular of a reflective material.

When the reflective particles do not exhibit a multilayer structure, they can be composed, for example, of metal oxides, in particular of titanium or iron oxides obtained synthetically.

When the reflective particles exhibit a multilayer structure, they can, for example, comprise a natural or synthetic substrate, in particular a synthetic substrate, at least partially coated with at least one layer of a reflective material, in particular of at least one metal or metal material. The substrate can be made of one or more organic and/or inorganic materials.

More particularly, it can be chosen from glasses, ceramics, graphite, metal oxides, aluminas, silicas, silicates, in particular aluminosilicates and borosilicates, synthetic mica and mixtures thereof, this list not being limiting.

The reflective material can comprise a layer of metal or of a metal material. [0090] Reflective particles are described in particular in the documents JP-A-09188830, JP-A-10158450, JP-A-10158541, JP-A-07258460 and JP-A-05017710.

Mention may also be made, still by way of example of reflective particles comprising an inorganic substrate coated with a layer of metal, of the particles comprising a borosilicate substrate coated with silver.

Particles comprising a glass substrate coated with silver, in the form of platelets, are sold under the name Microglass Metashine REFSX 2025 PS by Toyal. Particles comprising a glass substrate coated with nickel/chromium/molybdenum alloy are sold under the names Crystal Star GF 550 and GF 2525 by this same company.

Use may also be made of particles comprising a metal substrate, such as silver, aluminum, iron, chromium, nickel, molybdenum, gold, copper, zinc, tin, magnesium, steel, bronze or titanium, said substrate being coated with at least one layer of at least one metal oxide, such as titanium oxide, aluminum oxide, iron oxide, cerium oxide, chromium oxide, silicon oxides and mixtures thereof.

Mention may be made, by way of examples, of aluminum powder, bronze powder or copper powder coated with SiCk sold under the name Visionaire by Eckart.

Mention may also be made of pigments with an interference effect which are not attached to a substrate, such as liquid crystals (Helicones HC from Wacker) or interference holographic glitter (Geometric Pigments or Spectra f/x from Spectratek). Special effect pigments also comprise fluorescent pigments, whether these are substances which are fluorescent in daylight or which produce an ultraviolet fluorescence, phosphorescent pigments, photochromic pigments, thermochromic pigments and quantum dots, for example sold by Quantum Dots Corporation.

The various effect colorants which can be used in the present invention makes it possible to obtain a rich palette of colors and also specific optical effects, such as metallic effects or interference effects.

The size of the effect colorant used in the composition according to the present invention is generally between 10 nm and 200 microns, preferably between 20 nm and 80 microns and more preferentially between 30 nm and 50 microns.

In accordance with preferred embodiments of color-transforming compositions, the effect colorants (preferably pearlescent pigments) are preferably present in an amount from about 0.1% to about 2%, such as from about 0.2% to about 2% by weight, preferably from about 0.25% to about 1.5% by weight, preferably from about 0.5% to about 1.5%, of the total weight of the color-transforming composition, including all ranges and subranges therebetween.

According to certain embodiments, the color system of the color-transforming composition has a colorant fraction of effect colorants that is at least about 50%, preferably at least about 75%, preferably at least about 85%, preferably at least about 90%, preferably at least about 95%, such as 100%.

Suspending System

The color-transforming includes a suspending system consisting of one or more suspending agents. The suspending agent are useful for suspending colorants in the composition. The suspending system includes one or more suspending agents and specifically includes hydrophilic silica.

The hydrophilic silica may be a fumed silica, such as those having an average particle size less than 1 μm. By hydrophilic silica it is meant that the surface silanol groups are not chemically modified such as with trimethylsiloxyl groups.

The suspending system may optionally include other suspending agents including polymers having a good absorption affinity for the surface of pigments/colorants. According to certain embodiments the color-transforming composition (and therefore the suspending system) is substantially free of clays.

Clays are layered aluminosilicate materials and can be optionally be modified, in particular modified montmorillonite clays such as hydrophobic bentonites or hectorites modified such as hectorites modified with ammonium chloride in CIO to C22, such as hectorite modified with distearyl dimethyl ammonium chloride such as, for example, the product Disteardimonium Hectorite (name CTFA) (reaction product of hectorite and distearldimonium chloride) sold under the name Bentone 38 or Bentone Gel by the company Elementis Specialties. Mention may be made, for example, of the product Stearalkonium Bentonite (name CTFA) (reaction product of bentonite and quaternary ammonium stearalkonium chloride) such as the commercial product sold under the name TIXOGEL MP 250® by the company Sud Chemie Rheologicals, United Catalysts Inc.

In accordance with preferred embodiments, the suspending agents are preferably present in a total amount from about 0.5% to about 2.5% by weight, preferably from about 0.75% to about 2.5% by weight, and preferably from about 1% to about 2% by weight. Furthermore, the suspending system is preferably at least about 50% hydrophilic silica, such as at least about 60%, at least about 70%, such as at least about 80%, such as at least about 90%, such as 100% hydrophilic silica.

Film-Forming System

The color-transforming includes a film-forming system consisting of one or more film-formers (film-forming agents or film-forming polymers).

The film-forming system preferably includes a cellulose compound. Suitable cellulose compounds include, but are not limited to, cellulose polymers, such as, for example, hydroxyethylcellulose, hydroxypropylcellulose, methylcellulose, ethylhydroxyethylcellulose and nitrocellulose. Preferably the cellulose compound includes or consists of nitrocellulose.

According to preferred embodiments, the at least one cellulose compound, if present, is present in the compositions of the present invention in an amount of active material ranging from about 0.01 to about 30% by weight, more preferably from about 0.1 to about 20% by weight, and most preferably from about 5 to about 15% by weight, based on the total weight of the composition, including all ranges and subranges within these ranges.

Suitable film forming agents include water-soluble film forming agents and oil-soluble film forming agents.

Specific examples of suitable water-soluble film forming agents include, but are not limited to, latexes, proteins, keratins, for example keratin hydrolysates and sulfonic keratins; acrylic polymers or copolymers; vinyl polymers; gums; and their mixtures.

According to preferred embodiments, the at least one film forming agent is present in the compositions of the present invention in an amount of active material ranging from about 0.01 to about 30% by weight, more preferably from about 0.1 to about 20% by weight, and most preferably from about 5% to about 15% by weight, based on the total weight of the composition, including all ranges and subranges within these ranges.

Other Ingredients

One of ordinary skill in the art would readily understand the types of other ingredients typically found in nail compositions. A non-exhaustive list of such ingredients includes, but is not limited to plasticizing agents, coalescing agents, preservatives, fragrances, oils, waxes, surfactants, antioxidants, antifoaming agents, active principles chosen from essential oils, UV screening agents, sunscreens, moisturizing agents, vitamins, shine enhancing agents, shine-reducing agents, ceramides, plant extracts, fibers, and the like, and their mixtures.

Plasticizers (plasticizing agents) are additives used to optimize the mechanical properties of the films. They tend to reduce the Glass Transition Temperature (Tg) and increase the softness and flexibility of the films. Preferably, the plasticizer has a distribution coefficient D of less than or equal to 0.1.

Preferably, the plasticizer has a boiling point measured at ambient pressure of less than or equal to 285° C., preferably less than or equal to 270° C., and preferably less than or equal to 250° C. In the present specification, the boiling point values are to be considered accurate to ±2° C. owing to the uncertainties of boiling point measurement.

Any plasticizing agent typically found in nail polish compositions can be used. Examples of suitable plasticizers include, but are not limited to, glycols and their ester derivatives, esters of acids, in particular carboxylic acids, such as citrates, adipates, carbonates, tartrates, phosphates or sebacates, oxyethylenated derivatives, such as oxyethylenated oils, and their mixtures. For example, suitable plasticizing agents include, but are not limited to adipates, phthalates, citrates such as triethyl citrate, tributyl citrate, tributyl acetylcitrate, tri(2-ethylhexyl) acetylcitrate, and mixtures thereof.

In accordance with preferred embodiments, the plasticizer, if present, is preferably present in the primer composition in an amount of from 0.1% to 25% by weight, preferably from 1% to 10% by weight, preferably from 2% to 8% by weight, of the total weight of the composition, including all ranges and subranges therebetween.

Coalescents (coalescing agents) are additives used assist the film formation process of certain film forming agents (e.g., latex).

Any coalescent agent typically found in nail polish compositions can be used. Examples of suitable plasticizers include, but are not limited to, propylene glycol n-butyl ether, dipropylene glycol dimethyl ether, propylene glycol methyl ether acetate, propylene glycol propyl ether, methyl lactate, ethyl lactate, isopropyl lactate, and mixtures thereof.

In accordance with preferred embodiments, the coalescent agent, if present, may be present in the primer composition in an amount of from 0.1% to 25% by weight, preferably from 1% to 15% by weight, preferably from 3 to 10% by weight, of the total weight of the composition, including all ranges and subranges therebetween.

A person skilled in the art will take care to select the optional additional additives and/or the amount thereof such that the advantageous properties of the compositions according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 20% relative to the total weight of the composition and further such as from 0% to 10% or 0.5% to 5%, including all ranges and subranges therebetween.

Needless to say, the compositions of the invention should be cosmetically or dermatologically acceptable, i.e., they should contain a non-toxic physiologically acceptable. The compositions may be in any galenic form normally employed in the cosmetic and dermatological fields which is suitable for topical administration onto nails.

According to preferred embodiments, the color-transforming compositions of the present invention are anhydrous. According to certain other embodiments the color-transforming compositions of the present invention are phase stable, i.e., they show no visible phase instability after being held for one, more or all of: four weeks at 105° F., six weeks at 105° F., and/or three freeze thaw cycles.

As detailed above, according to certain embodiments, the color-transforming composition is applied onto the surface of nails having a decorative (first) coating having a substantially uniform color appearance formed thereon. This provides a transformation of the color appearance of the first coating. According to certain alternative embodiments, the color-transforming composition is applied instead directly to the nails. In this manner, the color-transforming composition acts to provide a translucent, nonuniform color appearance to the nail rather than to transform the color appearance of the first coating.

According to preferred embodiments of the present invention, a kit comprising (1) at least one color-transforming composition comprising (a) solvent; (b) a color system comprising one or more pigments, the one or more pigments comprising one or more pearl pigments, wherein the one or more pearl pigments are present in the color-transforming composition in a concentration by weight from about 0.1% to about 2%, and wherein the color system optionally comprises one or more additional colorants, the one or more additional colorants collectively present in a concentration by weight such that the colorant fraction of effect colorants is at least about 50% by weight; (c) a suspending system comprising one or more suspending agents, the one or more suspending agents comprising hydrophilic silica; and (d) a film-forming system, wherein the color-transforming composition is substantially free of clays; and

The kit further comprises, (2) at least one nail polish composition with a fraction of effect colorants as compared to a total concentration of colorants that is less than about 10%, such as less than about 5%, such as less than about 1%, such as less than about 0.5%, such as 0%.

The compositions according to the invention can be manufactured by known processes used generally in the cosmetics or dermatological field.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Examples

Decorative coatings were applied to artificial nails VITRO-NAILS (available from Florida Suncare Testing, Inc., IMS Division, Bunnell, Fla.) were coated with three different nail polish compositions and allowed to dry to form a decorative nail coating having a substantially uniform color appearance. The first decorative color coat formulation, EX1, (“color coat”) provided a red decorative nail coating. The second EX 2, provided a brown decorative nail coating. The third, EX3, provided a blue decorative nail coating. All three had substantially uniform color appearance. The three decorative color coat formulations included about 70% to about 75% of solvents; a color system comprising only conventional pigments, a film-forming system, plasticizers, and other ingredients typically used in nail polish formulations, such as sunscreens. The weight percentage of colorants (on a solids basis) included in each of the decorative color coat compositions are shown in Table 1 below

TABLE 1

| | EX 1 | EX 2 | EX 3 |
|---|---|---|---|
| Colorant | (Red) | (Brown) | (Blue) |
| Titanium dioxide | 0.986 | 1.58 | 0.222 |
| (and) oxidized | | | |
| polyethylene (and) | | | |
| aluminum hydroxide | | | |
| [white pigment] | | | |
| Rosinated red lake | 0.322 | 0.326 | 0 |
| 34 [red pigment] | | | |
| Rosinated Red lake | 0.321 | 0.326 | 0 |
| 6 [Red pigment] | | | |
| Ferric Ammonium | 0.0360 | 0 | 0.687 |
| Ferrocyanide | | | |
| [Blue pigment] | | | |
| Red Lake 7 | 0 | 0 | 0.261 |
| [Red pigment] | | | |
| Black 2 | 0 | 0.165 | 0.103 |
| [Black Pigment] | | | |
| TOTAL COLORANT | 1.67 | 2.40 | 1.27 |

After these decorative color coat formulations were applied and allowed to dry, a sample of each of these was then subsequently separately coated with each of three color transforming compositions, yielding 9 samples in total.

The color-transforming compositions had the following composition: as shown in Table 2:

TABLE 2

| INGREDIENT CLASS | CONCENTRATION BY WEIGHT (%) |
|---|---|
| Solvent | 60-80 |
| Film-Formers | 10-20 |
| Plasticizer | 4-10 |
| Suspending Agents | 1-2 |
| (hydrophilic silica only) | |
| Colorants (pearl pigments only) | 0.25-1.5 |
| Other ingredients | 0-5 |

The other ingredients included sunscreen and dimethicone.

The colorants included effect colorants (only pearlescent pigments) and, in certain cases, very small concentrations of conventional colorants. The pearlescent pigments used in the three color-transforming compositions are shown below in Table 3, below.

TABLE 3

| Colorant | EX 11 | EX 12 | EX 13 |
|---|---|---|---|
| Calcium borosilicate | 0.69 | | |
| (and) titanium dioxide | | | |
| (and) silica (and tin | | | |
| oxide | | | |
| Mica (and) titanium | 0.30 | | |
| dioxide (and) Carthaus | | | |
| tintorius flower extract | | | |
| (and) tin oxide | | | |
| Calcium aluminum | | | |
| borosilicate (and) silica | | | |
| (and)titanium | | | |
| dioxide(and) tin oxide | | | |
| Synthetic | | 0.40 | |
| fluorophlogopite (and) | | | |
| silica (and) titanium | | | |
| dioxide | | | |
| Synthetic | | 0.20 | |
| fluorophlogopite (and) | | | |
| silica (and) iron oxides | | | |
| Silica (and) titanium | | 0.40 | 0.40 |
| dioxide | | | |
| Silica and synthetic | | | 0.10 |
| fluorophlogopite and | | | |
| titanium dioxide and | | | |
| carbon black | | | |
| TOTAL COLORANTS | 0.99 | 1.00 | 0.50 |

The colorant fraction of effect colorants was approximately 100%. The color-transforming compositions were substantially free of clays and were anhydrous. All of the color-transforming compositions transformed the color-appearance of the decorative nail coating from a uniform color appearance to one that varied as a function of the conditions of observation. The color transformations are noted in the Table 4, below:

TABLE 4

| | | Color | | |
|---|---|---|---|---|
| | Decorative | Transforming | Initial Color | Final Color |
| Reference | Nail Coating | Composition | Appearance | Appearance |
| Ex 21 | Ex 1 | Ex. 11 | Uniform Red | Non-Uniform: |
| | | | | Grainy gold |
| | | | | and red |
| Ex 24 | Ex 1 | Ex. 12 | Uniform Red | Non-Uniform: |
| | | | | Grainy gold |
| | | | | and red |
| Ex 25 | Ex 1 | Ex. 13 | Uniform Red | Non-Uniform: |
| | | | | Grainy silver |
| | | | | and red |
| Ex 26 | Ex 2 | Ex. 11 | Uniform Brown | Non-Uniform: |
| | | | | Grainy gold |
| | | | | and brown |
| Ex 27 | Ex 2 | Ex. 12 | Uniform Brown | Non-Uniform: |
| | | | | Grainy gold |
| | | | | and brown |
| Ex 28 | Ex 2 | Ex. 13 | Uniform Brown | Non-Uniform: |
| | | | | Grainy |
| | | | | silver/gold |
| | | | | and brown |
| Ex 29 | Ex 3 | Ex. 11 | Uniform Blue | Non-Uniform: |
| | | | | Grainy gold |
| | | | | and blue |
| Ex 30 | Ex 3 | Ex. 12 | Uniform Blue | Non-Uniform: |
| | | | | Grainy gold |
| | | | | and blue |
| Ex 31 | Ex 3 | Ex. 13 | Uniform Blue | Non-Uniform: |
| | | | | Grainy |
| | | | | silver/gold |
| | | | | and blue |

Surprisingly, the background color of the decorative nail coat is not blended away or completely hidden by the color-transforming composition. Rather brand new color appearances are achieved.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

What is claimed is:

1. A method of transforming the color appearance of a nail, comprising the steps of:
applying a color-transforming composition to a nail having a decorative coating with substantially uniform color appearance formed thereon, wherein the color-transforming composition comprises:
solvent;
a color system comprising one or more colorants, the one or more colorants comprising one or more effect colorants, wherein the one or more effect colorants are present in the color-transforming composition in a concentration by weight from about 0.1% to about 2%, and wherein the color system optionally comprises one or more additional colorants, wherein the color system has a colorant fraction of effect colorants that is at least about 50% by weight;
a suspending system comprising one or more suspending agents, the one or more suspending agents comprising hydrophilic silica; and
a film-forming system, wherein the color-transforming composition is substantially free of clays.

2. The method of claim 1 wherein the decorative coating having substantially uniform color appearance has a colorant fraction of effect colorants less than about 10%.

3. The method of claim 1 wherein the decorative coating having substantially uniform color appearance has a colorant fraction of effect colorants less than about 1%.

4. The method of claim 1 wherein the color system of the color-transforming system has a colorant fraction of effect colorants that is at least about 85%.

5. The method of claim 1 wherein the color system of the color-transforming composition has a colorant fraction of effect colorants that is at least about 95%.

6. The method of claim 1 wherein the color-transforming composition comprises from about 0.25% to about 1.5% by weight of effect colorants.

7. The method of claim 1 wherein the color-transforming composition comprises from about 0.5% to about 1.5% by weight of effect colorants.

8. The method of claim 1 wherein the one or more effect colorants comprise pearlescent pigments.

9. The method of claim 1 wherein the one or more effect colorants consist of pearlescent pigments.

10. The method of claim 1 wherein the color-transforming composition comprises from about 0.25% to about 1.5% by weight of pearlescent pigments.

11. The method of claim 4 wherein the color-transforming composition comprises from about 0.25% to about 1.5% by weight of pearlescent pigments.

12. The method of claim 5 wherein the color-transforming composition comprises from about 0.25% to about 1.5% by weight of pearlescent pigments.

13. The method of claim 1, wherein the color-transforming composition transforms the color appearance of the decorative coating to a non-uniform color appearance that varies as a function of conditions of observation.

14. The method of claim 1, wherein the decorative coating is formed by applying a nail polish composition having a colorant fraction of effect colorant that is less than about 10% by weight.

15. A nail composition, comprising
about 50% to about 85% by weight of one or more solvents;
a color system comprising one or more colorants, the one or more colorants comprising one or more pearlescent pigments, wherein the one or more pearlescent pigments are present in the nail composition in a concentration by weight from about 0.1% to about 2%, and wherein the color system has colorant fraction of pearlescent pigments that is at least about 90% by weight;
a suspending system comprising at least about 80% hydrophilic silica; and
a film-forming system, wherein the nail composition is substantially free of clays.
